(19) United States
(12) Patent Application Publication
(10) Pub. No.: US 2015/0173594 A1
(43) Pub. Date: Jun. 25, 2015

(54) Method and Device for Improved Hygiene During Endoscopic Procedures

(71) Applicant: Ashkan Farhadi, Irvine, CA (US)

(72) Inventor: Ashkan Farhadi, Irvine, CA (US)

(21) Appl. No.: 14/027,245

(22) Filed: Sep. 16, 2013

Related U.S. Application Data
(60) Provisional application No. 61/847,984, filed on Jul. 18, 2013.

(51) Int. Cl. A61B 1/00

(52) U.S. Cl. CPC A61B 1/00142; A61B 1/00137

(57) ABSTRACT

An elongated flexible sleeve over the endoscope shaft and secured to the endoscope shaft at the proximal portion protects the shaft of the endoscope from touching the external objects before the endoscope shaft is inserted into a body cavity and after the endoscope shaft is withdrawn from the body cavity. In use, the endoscope cover stays outside of the body cavity at the proximity of the body cavity opening at all times. The sleeve may be supplied with a dispenser that releases a lubricant over the endoscope shaft at the tip of the cover during endoscope insertion. The distal end portion of the endoscope cover also may be supplied with an absorbent liner to wipe off secretions and lubricant during endoscope withdrawal.

### CROSS-REFERENCE TO RELATED APPLICATION

This application claims the benefit of priority to Provisional patent application 61/847,984 entitled “ Endoscope Cover ” filed on Jul. 18, 2013. The contents of the above-listed patent application are hereby incorporated by reference in its entirety.

### FIELD OF INVENTION

This invention relates generally to methods and devices that provide for improved hygiene in the endoscopic examination of body organs, particularly the gastrointestinal tract. The invention relates to providing a cover for an endoscope that remains outside of the patient's body during an endoscopic exam that is configured to keep the endoscope clean prior to insertion into the patient's body, and to retain any bodily matter within upon removal of the endoscope from the patient's body.

### BACKGROUND OF INVENTION

An endoscope is a well-known optical system for evaluation of internal organs that was disclosed and claimed in U.S. Pat. No. 3,449,037 to C. J. Koester. Currently used fiber optic endoscopes are comprised of many lenses mounted in a flexible tube to relay an image from inside a body cavity for viewing by a physician for diagnosis or manipulation inside cavitary spaces. Most endoscopic procedures are not sterile procedures. After completion of a cleansing process, the endoscope is handled as a clean object; usually kept in clean spaces, transported to the procedure room by clean hand with or without gloves, placed on a clean surface for assembly to the light source, (placed on bed sheet in the case of colonoscopy) and then lubricated and inserted into a body cavity. During the procedure the shaft of the endoscope is usually grasped by the endoscopist for insertion, withdrawal, and manipulation. The endoscope could be inserted and withdrawn multiple times, and each time the shaft of the endoscope touches the external environment including the bedding, examining table and patients external body surfaces. The procedures that involves biopsy taking or other endoscopic interventions usually provide further hands and surfaces to be touched during and after the procedure. After withdrawal from a body cavity, the endoscope is handled as a contaminated object. It is handled with care, and taken to the cleaning room where the endoscope is cleaned inside and out to be used for the next procedure. To avoid contamination of the endoscope and endoscopic room and devices and to improve the sanitization of the endoscopic procedures, there are several policies and techniques in place governed by sanitizing agencies.

Darras in 1989 (Medical instrument cover, U.S. Pat. No. 4,886,049) disclosed a medical instrument cover having an elongated, generally tubular sheath of an elastomeric material, open at both ends, configured to fit on an endoscope having a filament in its interior surface to separate the sheath in an elongate direction and two strips to facilitate the removal of the cover from the endoscope by pulling open the sheath along its length. The designed cover is made from “an elastomeric material to ensure a snug and intimate fit of the sheath on the medical instrument” The sheath is inserted with the endoscope into a body cavity and the inventor suggested “it may be desirable to employ a lubricating agent along the outside of the sheath”. The cover has also a “means for removing the sheath from the medical instrument using a filament while in the body or two stripes while outside of the body”.

Another device is disclosed by Chikama et. Al., U.S. Pat. No. 5,159,919. This device is an endoscope cover comprising a soft disposable cylindrical cover which covers the rigid distal end of an endoscope during endoscope insertion to protect the exposure of the endoscope distal end to the body cavity and improve the cleaning and disinfection process of the endoscope. The cover has a transparent window arranged on a closed face formed on its distal rigid end. The rigid cover is tightly fitted; it is difficult to dismount the rigid cover. The primary object of the invention is to protect the endoscope from contamination during use and to permit immediate reuse of the endoscope.

Another device was disclosed by Hickes, U.S. Pat. No. 5,198,894. This is a “Drape for endoscope” and is an endoscope sleeve-like drape secured in a retracted position at the proximal end of an endoscope. The drape is extended to telescope over and envelope the endoscope CCD camera portion such that the resulting outer surface of the drape in its extended position remains sterile. This device is designed to keep the camera at the end of the endoscope sterile but is not designed for covering the endoscope shaft.

Another device was disclosed in U.S. Pat. No. 5,569,161 by Ebling, et al. in 1996 called “Endoscope with sterile sleeve”. This device has a sterile sleeve that accommodates endoscope fiber optic bundle to avoid the need for sterilizing fiber optic prior to use. A window at the distal end of the sleeve allows viewing through the end of sleeve.

Yet another device was disclosed in U.S. Pat. No. 6,293,907 by Axon, et al called “Endoscope cover having protrusions”. This is a removable cover for an endoscope shaft that includes a sleeve of elastic material having a low friction external surface to improve the frictional resistance to forward movement of the endoscope shaft compared to frictional resistance to reverse movement of the endoscope shaft. The friction between the sleeve and shaft is as such that it prevents relative longitudinal movement of the sleeve and endoscope shaft without requiring the sleeve to be gripped. If desired a lubricant can be applied to the exterior of the sleeve. The frictional resistance differs in the forward and reverse directions, which correspond in use to the insertion and withdrawal direction. The sleeve is inserted with the endoscope shaft inside the body and results in selective pulling of the bowel over the endoscope even when withdrawing force is applied to the endoscope shaft, which in turn facilitates the reduction in loops in the bowel by causing the bowel to pull itself over the endoscope.

Another device is disclosed in U.S. Pat. No. 6,852,077 by Ouchi, et al in 2005 entitled “Cover for preventing contamination of an operating portion of an endoscope”. This is a cover for preventing contamination of an operating portion of an endoscope that is not inserted within the body. The cover is formed in a bag-like shape for enveloping the operating portion by forming a hole for passing the endoscope shaft within the cover on one side and another larger opening in a sleeve-like manner for entering a hand to operate the endoscope operating portion (knob). This device does not cover the endoscope shaft at all.

Another device is disclosed in U.S. Pat. No. 8,262,561 invented by Kress in 2012 entitled “Hygiene protection for endoscopes”. This is a cover for endoscope shaft which is closed at its distal end and is transparent for optical information. It can be rolled up over the shaft of an endoscope in a condom manner in the direction of the axis of the endoscope and includes one or more working and vacuum channels extending in a parallel position in relation to the endoscope and terminating in an open manner on the distal end of the cover. The working channels are only connected to the distal end of the cover but vacuum channel create a sub-atmospheric pressure within the cover so the cover adhere tightly to the endoscope shaft for insertion into body. The cover also has a transparent pane or lens at its said distal end that allows the distal end of the endoscope to face this end window for acquiring images within the body cavity. Again this cover enters the body cavity over the endoscope shaft.

### SUMMARY OF THE INVENTION

The endoscope cover of the present invention is intended to cover a variety of medical instruments to improve the endoscopic sanitization. Exemplary of the medical instruments which are intended to be covered by the cover of the current application are endoscopes. Those skilled in the art will recognize that the term endoscope refers to an instrument for visualizing the interior of a hollow organ. Specifically, the covering of the present invention is suitable for use on sigmoidoscopes, duodenoscopes, upper G.I. fiberscopes, bronchofiberscopes, colonoscopes and the like.

The endoscope cover embodying this invention enhances endoscopic procedure sanitization and facilitates endoscopic procedure in general and to this end the device is comprised of an elongated flexible sleeve sized to receive and endoscope shaft and having a length sufficient to extend beyond full length of an endoscope shaft, having a proximal opening and a distal opening, configured to define a passageway for an endoscope shaft and encloses an endoscope shaft in its entire length. The cover interior diameter is larger than the endoscope exterior diameter so the cover easily accommodates the endoscope shaft. The cover proximal opening can be pulled over the distal end of an endoscope shaft and be pulled proximally along the endoscope shaft till it reaches the proximal portion of the endoscope shaft adjacent to the endoscope control knob. The cover proximal end portion can then be secured to the endoscope shaft at this portion using an elastic end portion or an adhesive. The length of the endoscope cover is as such that after securing the proximal end portion of the cover to the proximal portion of the endoscope shaft, the distal end portion of the endoscope may still stay within the lumen of the endoscope cover. The interior surface of the mid-portion of the endoscope cover is made from a material that creates minimal friction between the internal surface of the endoscope cover and the exterior surface of the endoscope and can be reversibly pleated over the shaft of the endoscope. A handle at the distal end portion for positioning the distal end of the sleeve relative to the distal end of the endoscope shaft. The endoscope shaft can be grasped through handle portion of the cover. The material of the handle is configured to create adequate friction between the interior surface of the grasping handle and the exterior surface of the endoscope when it is being grasped to allow for endoscope shaft insertion, withdrawal, or manipulation.

In action, the cover is pulled over the endoscope shaft and secured to the endoscope shaft at the proximal portion just after the cleaning of the endoscope and before any handling or transportation of the endoscope to the examining room. When the endoscope needs to be 130 inserted into body cavity, the distal tip of the endoscope is extended beyond the cover by pulling the cover the endoscope shaft proximally. The endoscope shaft can be handled throughout the procedure while the shaft is being grasped over the grasping handle of the endoscope cover. The endoscope cover stays outside of the body cavity at the proximity of the body cavity opening at all times. During the insertion of the endoscope into the body cavity the endoscope cover distal end portion is pulled over the endoscope shaft proximally to allow adequate exposure of the portion of the endoscope shaft that needs to be inserted into the body cavity. This maintains the distance of the distal opening of the endoscope cover at the proximity but outside of the opening of the body cavity. During the withdrawal of the endoscope shaft from the body cavity, the endoscope cover is pulled over the endoscope shaft distally to cover the endoscope shaft out of the body. This maintains the distance of the distal opening of the endoscope cover at proximity but outside of the opening of the body cavity. At the termination of the endoscope shaft withdrawal from the body cavity, the cover is pulled all the way over the endoscope shaft to completely enclose the entire length of the endoscope shaft. During use, the endoscope cover prevents any contact of the endoscope shaft with endoscopist gloves as well as external surfaces to reduce the contamination of the endoscope shaft and during withdrawal the endoscope cover prevents any contact of the endoscope shaft with endoscopist gloves as well as external surfaces to reduce the contamination of the endoscopist gloves as well as external surfaces.

Therefore, it is an object of this invention to provide an extracorporeal endoscope cover comprising an elongated, flexible sleeve sized to receive an endoscope shaft and having a length sufficient to extend beyond full length of an endoscope shaft with a proximal end, and a distal end, configured to cover the shaft of an endoscope in a loose slidable relationship, for the purpose of insuring cleanliness of the endoscope prior to use, and to contain bodily matter disposed upon the endoscope within, following use.

In accordance with one aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the 160 endoscope shaft, whereby the distal end of the cover is configured to extend beyond the distal end of the endoscope prior to and following use, and is configured to be withdrawn proximal to the distal end of the endoscope during endoscopic use while remaining outside of the patient's body.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the proximal collar comprises an elastomeric cylindrical structure with an inner diameter larger than the insertion portion of the endoscope shaft, and smaller than the endoscope shaft at the intended fixation portion of the endoscope shaft.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the handle comprises an elastomeric cylindrical structure with an inner diameter larger than the insertion portion of the endoscope shaft, and is configured for translating user grasping forces to the shaft of the endoscope for the purpose of procedural manipulation of the endoscope shaft.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the handle comprises an endoscopic lubricant dispenser configured lubricating the endoscope shaft in order to facilitate endoscope insertion.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein an absorbent material is disposed upon the inner surface of the handle and is configured for removing lubricant or bodily matter from the endoscope shaft as it is withdrawn from the patient's body.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein an annular squeegee is disposed within the handle and is configured for removing lubricant or bodily matter from the endoscope shaft as it is withdrawn from the patient's body.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein an annular squeegee is disposed within the handle and is configured for removing lubricant or bodily matter from the endoscope shaft as it is withdrawn from the patient's body, and an annulus of absorbent material is disposed within the handle proximal to the annular squeegee and is configured for absorbing residual lubrication from the surface of the endoscope shaft in order to facilitate grasping and surgical manipulation endoscope shaft by the operator. In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein an annular squeegee is disposed within the handle and is configured for removing lubricant or bodily matter from the endoscope shaft as it is withdrawn from the patient's body, and an annulus of absorbent material is disposed within the handle proximal to the annular squeegee and is configured for absorbing residual lubrication from the surface of the endoscope shaft in order to facilitate grasping and surgical manipulation endoscope shaft by the operator, and a lubricant dispenser configured for applying a lubricant to the distal end of endoscope shaft by user squeezing disposed on the inner wall of the handle, and distal to the squeegee.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the elongated membranous structure comprises an extruded thin walled polyethylene sleeve, or similar structure, with an inner diameter larger than the insertable portion of the endoscope shaft.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the length of the elongated membranous structure exceeds the length of the insertable portion of the endoscope with a length sufficient for forming a prolapsed seal over the distal end of the cover.

In accordance with another aspect of this invention is an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body, wherein the cover further comprises a removable end-cap configured for enclosing the distal end prior to, and following endoscope use.

In addition, a method is disclosed here within for improved hygiene in the endoscopic examination of a patient's body comprising the steps of cleaning and sanitizing an endoscope for use, then covering the insertable portion of the endoscope shaft with an extracorporeal endoscope cover comprising an elongated flexible sleeve with a proximal end, and a distal end; with the proximal end comprising a collar configured for slidable engagement or fixation with a proximal portion of an endoscope shaft; and the distal end comprising a handle configured for grasping the endoscope shaft, whereby the distal end of the cover is configured to extend beyond the distal end of the endoscope prior to and following use, and is configured to be withdrawn proximal to the distal end of the endoscope during endoscopic use while remaining outside of the patient's body; then bringing the covered endoscope to the procedure room, then assembling the light source to the endoscope, then sliding the distal end of the endoscope cover in the proximal direction to uncover the distal end of the endoscope shaft, then inserting the endoscope into the patient and performing an endoscopic examination or surgical procedure, then withdrawing the endoscope from the patient and into the endoscope cover until the distal end of the endoscope is fully contained within the cover, then bringing the covered endoscope to a cleaning facility for cleaning and sanitation, whereby the use of the endoscope cover as disclosed keeps the endoscope clean prior to use, and prevents bodily matter disposed upon the endoscope after use from contaminating the workplace.

### BRIEF DESCRIPTION OF FIGURES

FIG. 1 is a schematic illustration showing the endoscope cover installed over the shaft of an endoscope with the distal end of the endoscope extending beyond the distal end of the endoscope cover.

FIG. 2 is a schematic illustration showing the endoscope cover installed over the shaft of an endoscope with the distal end of the endoscope cover extending beyond the distal end of the endoscope.

FIG. 3 is a cross sectional view taken at Section A-A in FIG. 1.

FIG. 4 is a cross sectional view taken at Section B-B in FIG. 1.

FIG. 5 is a longitudinal cross sectional view taken at Section C-C in FIG. 2.

FIG. 6A is a cross sectional view of the proximal end of the endoscope cover positioned over the the proximal insertion portion of an endoscope. FIG. 6B is a cross sectional view of the proximal end of the endoscope cover fixated to the proximal end of an endoscope shaft.

FIG. 7A is a schematic illustration of the distal end of the endoscope cover, covering the shaft of an endoscope with an end cap covering the distal end of the endoscope cover. FIG. 7B is a schematic illustration showing the end cap removed.

FIG. 8 is a schematic illustration of the endoscope cover, covering an endoscope, with the elongated sleeve of the endoscope cover prolapsed over the distal end of the endoscope cover enclosing the distal end of the endoscope cover.

FIG. 9A is a schematic illustration of the distal end of an endoscope cover comprising a handle with an annular squeegee and annulus of wiping material disposed on the inner surface. FIG. 9B is a cross sectional view taken at Section D-D in FIG. 9A. FIG. 9C is a cross sectional view taken at Section E-E in FIG. 9A. FIG. 9D is a cross sectional view of an alternative embodiment of the handle further comprising a lubricant dispenser.

### DETAILED DESCRIPTION OF THE INVENTION

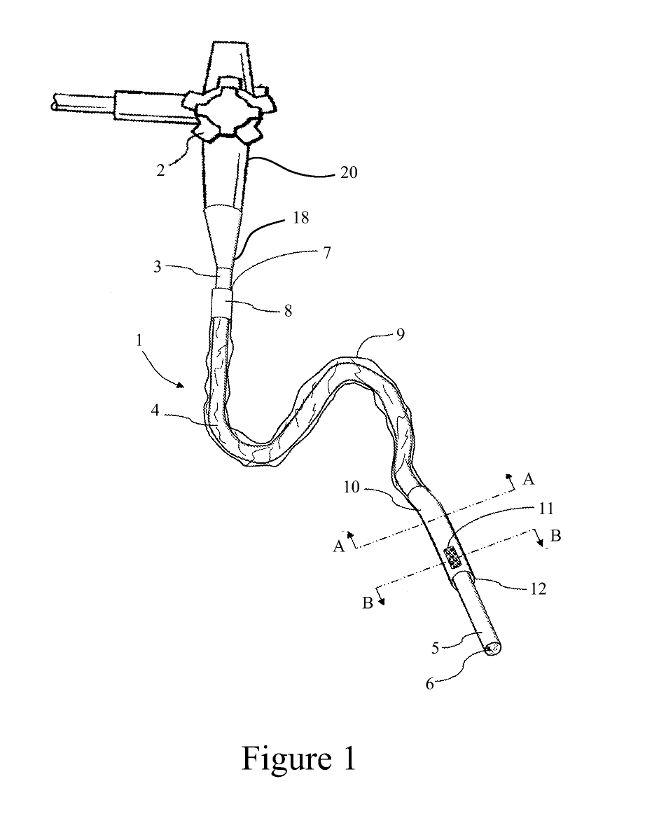

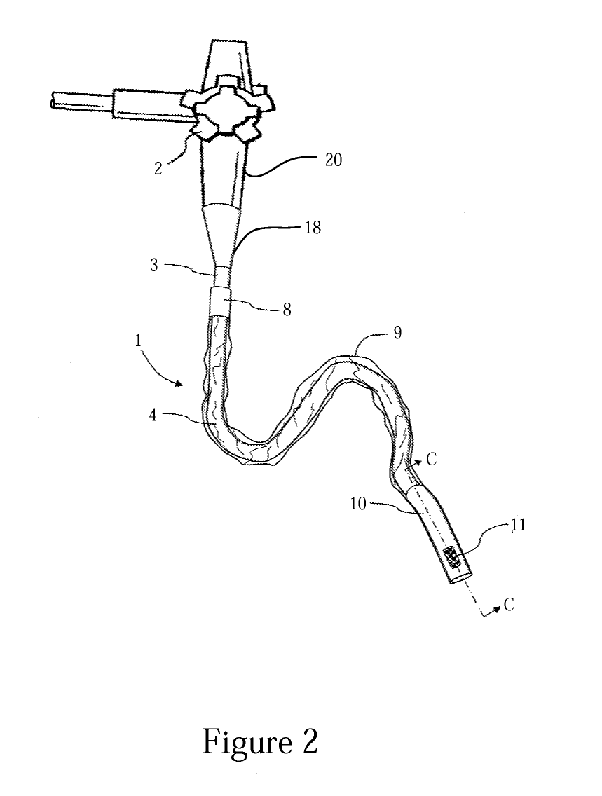

As depicted in FIG. 1 and FIG. 2, endoscope cover 1 comprises endoscope cover sleeve 9, proximal collar 8, disposed on the proximal end of sleeve 9, and handle 10 disposed on the distal end of sleeve 9. Endoscope cover 1 is an elongated flexible sleeve, having an open proximal end 7 associated with proximal collar 8 and a distal opening 12, associated with handle 10 as shown. Endoscope cover 1 is configured to fit over endoscope shaft 4, defining a passageway for endoscope shaft 4. Sleeve 9 has an interior diameter larger than the exterior diameter of endoscope shaft 4 so that endoscope cover 1 can easily accommodates endoscope shaft 4. Endoscope cover 1 proximal opening 7 can be pulled over distal end portion 5 of endoscope shaft 4 and pulled proximally along the endoscope shaft 4 until proximal opening 7 reaches the proximal portion 3 of endoscope shaft 4 adjacent to endoscope shaft boot 18, which is adjacent to control module 20 comprising operation knob 2. Endoscope cover 1 proximal collar 8 can then be secured to the endoscope shaft proximal portion 3 using an interference fit, or an adhesive, which will be described in further detail below. The length of endoscope cover 1 is such that after securing proximal collar 8 to proximal portion 3 of the endoscope shaft 4, distal opening 12 extends beyond the end 6 of endoscope shaft 4 as shown in FIG. 2. Handle 10 is disposed at the distal end of sleeve 9 and is configured for grasping endoscope shaft 4 for purpose of insertion, withdrawal, or manipulation without physically touching endoscope shaft 4. Sleeve 9 may comprise an extruded membrane of polymeric material such as thin walled polyethylene which may have a wall thickness between approximately 0.1 mm and 0.5 mm, with an inner diameter which may be between approximately 25% and 50% larger than the diameter of endoscope shaft 4. Sleeve 9 may be fabricated from alternative polymeric material providing that the interior surface of sleeve 9 is made from a material that creates minimal friction to the exterior surface of the endoscope shaft 4 and can be reversibly be pleated over the endoscope shaft 4 as handle 10 of the endoscope cover 1 is moved in the proximal direction as endoscope shaft 4 is inserted into a patient orifice. Handle 10 is a hollow cylindrical structure made from an elastomeric material such silicone rubber, or a urethane rubber with an inner diameter which may be between approximately 5% to 15% larger than the outer diameter of endoscope shaft 4, with a wall thickness which may be between approximately 2 mm and 6 mm, with a length which may be between approximately 5 cm and 15 cm. Handle 10 may be made from another elastomeric material, or non-elastomeric material providing the internal surface of handle 10 has adequate friction to endoscope shaft 4 when it is being grasped to allow insertion, withdrawal and manipulation of endoscope shaft 4.

Just before the insertion of the endoscope into a body cavity, handle 10 is pulled over the endoscope shaft proximally to allow the distal end of the endoscope 6 extend beyond the distal opening 12. The endoscope can then be inserted into a body cavity while the endoscope shaft is being grasped over handle 10. Handle 10 stays at the proximity but outside of the body cavity opening at all times. During insertion of endoscope handle 10 is pulled over the endoscope shaft 4 proximally to allow adequate exposure of the endoscope shaft that needs to be inserted into the body cavity. This maintains the relative distance of the distal opening of the endoscope cover 12 outside of the opening of the body cavity. During withdrawal of endoscope shaft 4 from the body cavity, handle 10 is pulled over the endoscope shaft 4 distally as the endoscope is being pulled out of the body cavity. This maintains the relative distance of the distal opening 12 outside of the opening of the body cavity. When the withdrawal of the endoscope from the body cavity is complete, handle 10 is pulled completely over endoscope distal end 6.

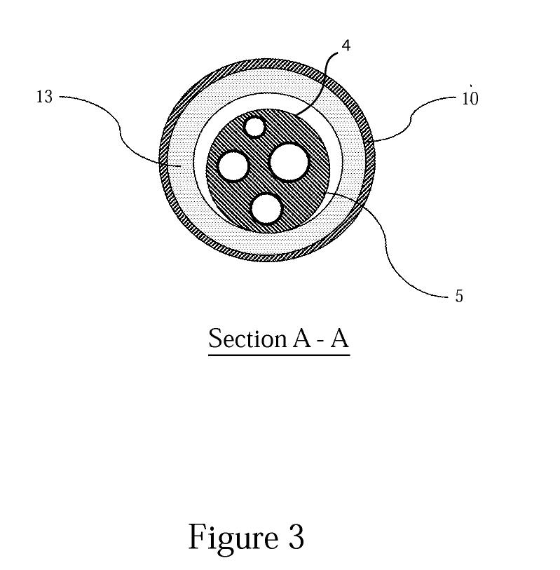

FIG. 3 is a cross sectional view taken at Section A-A of FIG. 1. As shown handle 10 encompasses distal portion 5 of endoscope shaft 4. Also shown is wiping material 13 disposed on the interior of handle 10. Wiping material 13 is configured for absorption of bodily matter, or endoscope lubricant as endoscope shaft 4 and handle 10 move in slidable relationship. Wiping material 13 may be a hydrophilic foam, or flox, or may be another absorbent material.

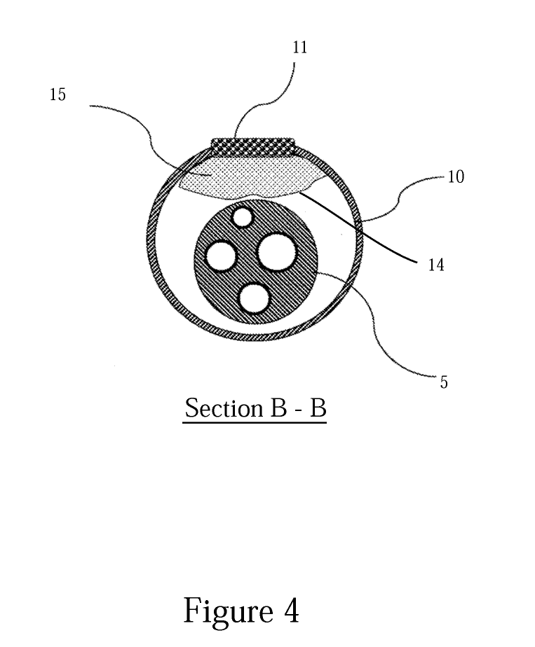

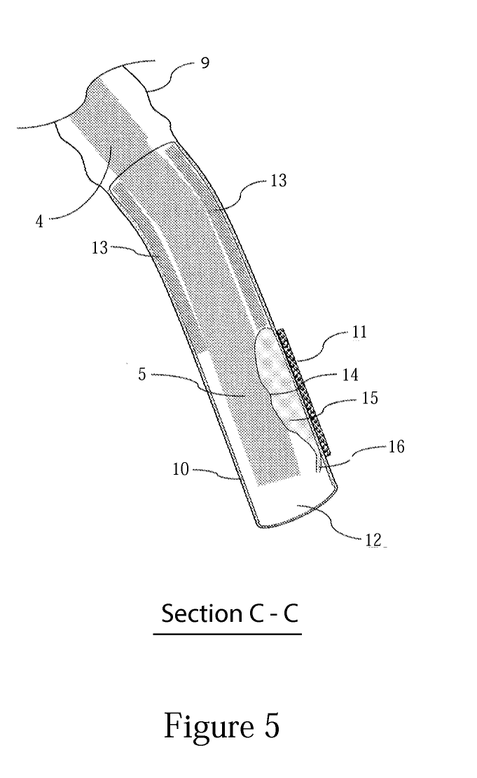

FIG. 4 and FIG. 5 are a cross sectional views taken at Section B-B of FIG. 1, and Section C-C of FIG. 2 respectively showing an optional embodiment where handle 10 is configured with a lubricant gel dispenser comprising lubrication gel reservoir 14, and lubrication dispensing button 11. Lubrication gel reservoir 14 holds lubricant material 15 at the distal interior surface of handle 10 as shown. Pressing the button 11 squeezes lubrication gel reservoir 14 against endoscope shaft 4 resulting in the release of the lubricant material 15 from the small opening 16 at the distal end of lubrication gel reservoir 14, and over the external surface of the endoscope shaft 4. This provides endoscope shaft 4 with lubrication during the insertion of endoscope shaft 4 into the patient.

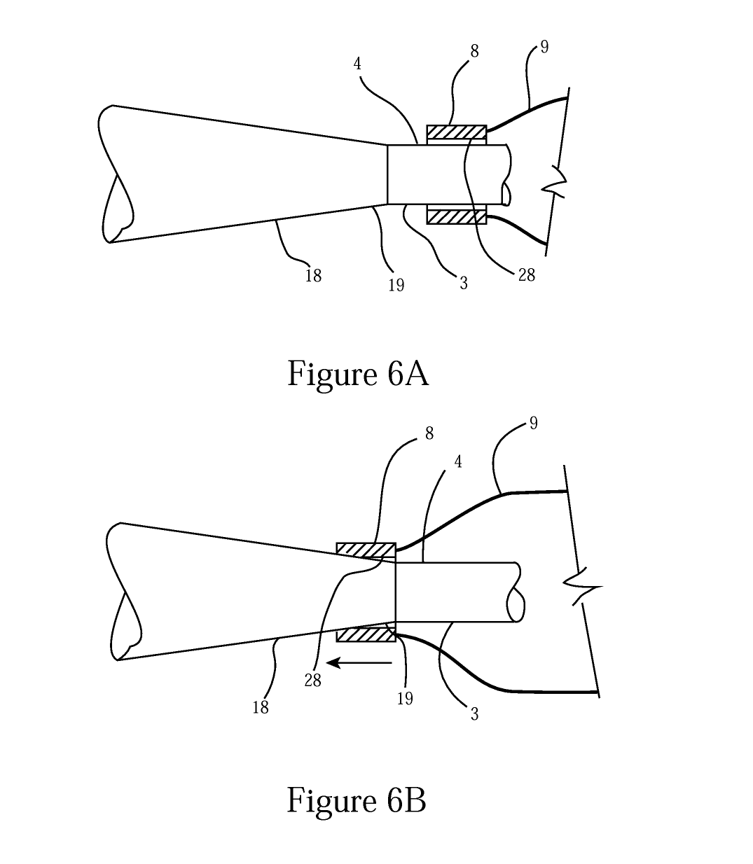

FIG. 6A is a cross sectional view of the proximal end of the endoscope cover 1 positioned over distal portion 3 of endoscope shaft 4 showing proximal collar 8 adjacent and distal to endoscope shaft boot 18, which connects endoscope shaft 4 to endoscope control module 20. As seen, the interior diameter 28 of proximal collar 8 is larger than endoscope shaft 4 to facilitate easy placement of endoscope cover 1 over endoscope shaft 4. FIG. 6B is a cross sectional view of the endoscope cover 1 proximal collar 8 fixated at the proximal end of an endoscope shaft 4 using an interference fit between the inner diameter 28 of proximal collar 8 and transition portion 19, of endoscope shaft boot 18. Proximal collar 18 may be fabricated from an elastomeric material with a high coefficient of friction. The combination of a high coefficient of friction, and elastic deformation of internal diameter 28 of proximal collar 8 provides sufficient fixation strength between endoscope shaft boot 18 and proximal collar 8. Those skilled in the art of medical devices are familiar with alternative means for fixating the proximal end of endoscope cover 1 to the proximal region of endoscope shaft 4 and remain within the scope of this invention.

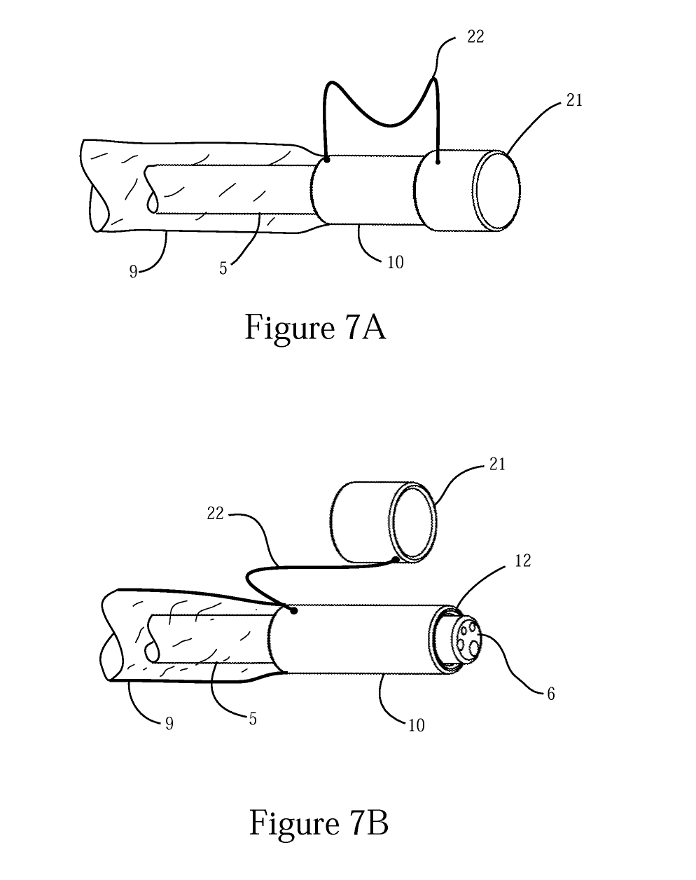

FIG. 7A and FIG. 7B depict an alternate embodiment of endoscope cover 1 comprising removable end cap 21, and optional end cap tether 22. As shown end cap 21 covers distal opening 12, as shown in FIG. 7B, and can be removed during endoscope use, and then replaced following endoscope use. End cap 21 can be configured for an interference fit with the distal end of handle 10 as shown, or may use a threaded interface with handle 10 or another means of removably mating end cap 21 to handle 10 as shown.

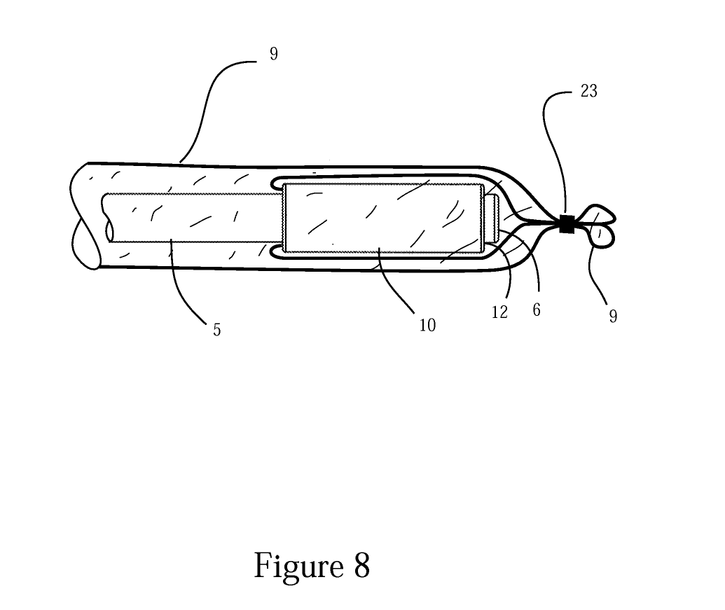

FIG. 8 depicts an alternate means of closing distal end 12 showing sleeve 9 being pulled distal in a prolapsed configuration as shown, with removable closure band 23 holding sleeve 9 in its prolapsed position. The closure method as shown requires that sleeve 9 be sufficiently long to form the prolapsed closure. Removable closure band 23 may be a rubber band, a twist tie, or another fastening device. The prolapse closure depicted, may be used prior to endoscope use, as well as following endoscope use.

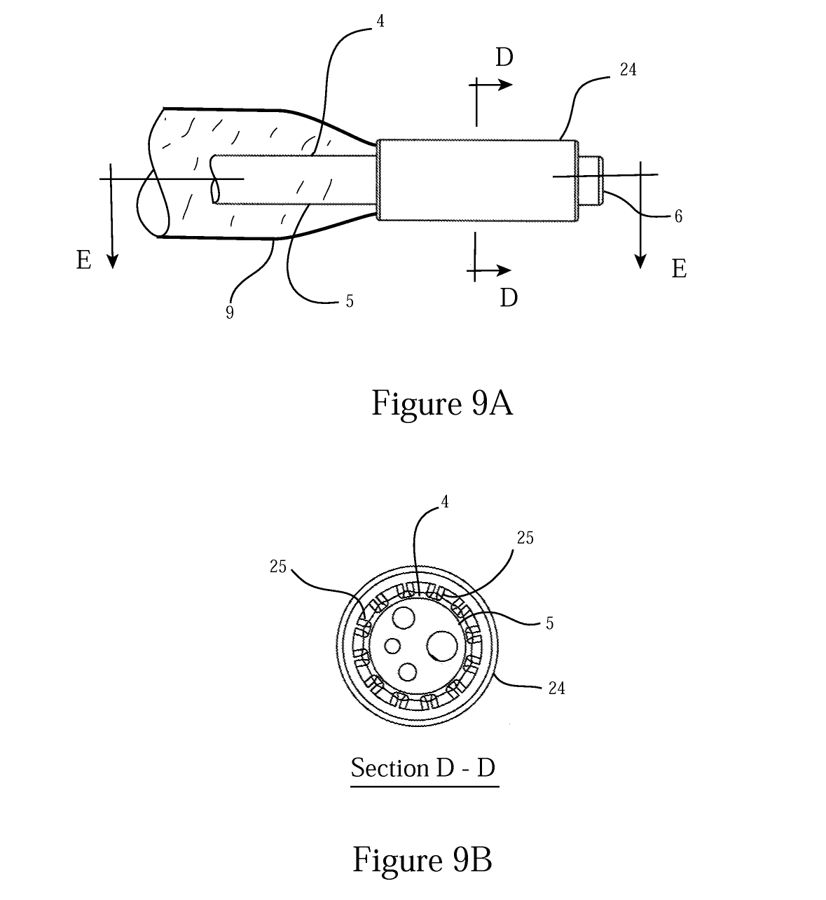

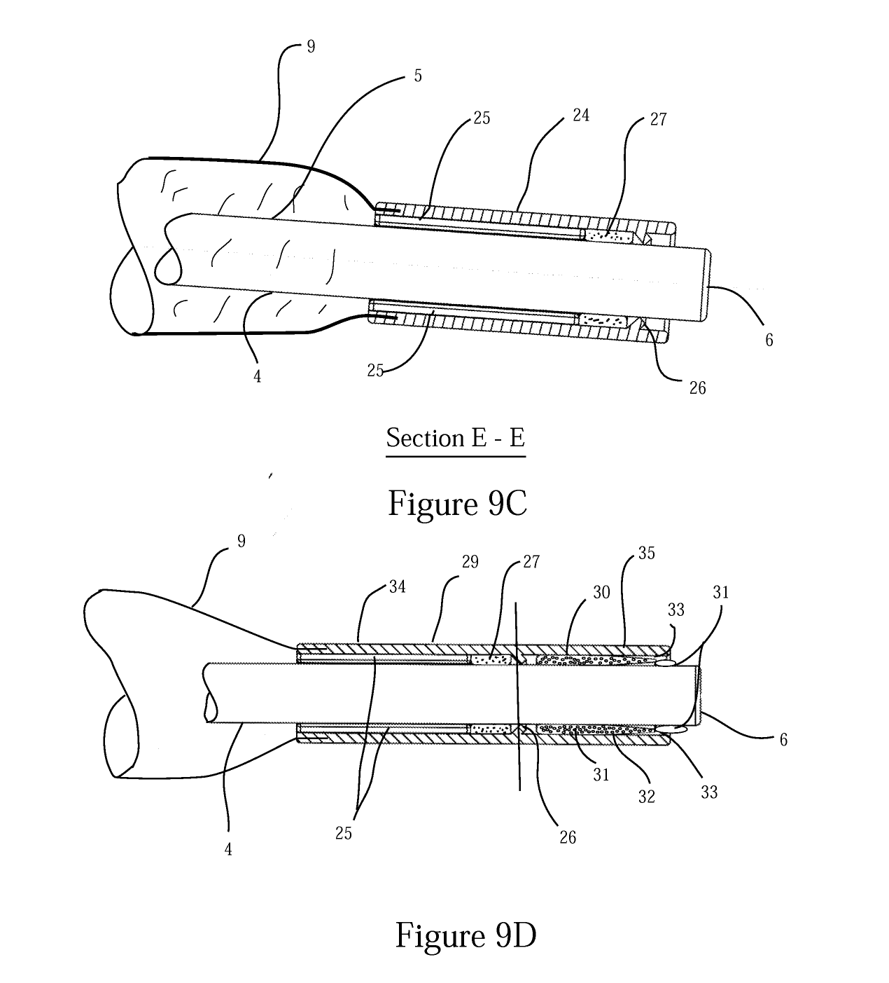

FIG. 9A is a schematic illustration of an alternative embodiment of the distal end of endoscope cover 1 comprising handle 24 with an annular squeegee and an annulus of wiping material disposed on the inner surface. FIG. 9B is a cross sectional view taken at Section D-D in FIG. 9A. FIG. 9C is a cross sectional view taken at Section E-E in FIG. 9A. As shown in in FIG. 9C, Section E-E, annular squeegee 26 is disposed on the inner surface of handle 24 near its distal end. Annular squeegee 26 is a protrusion from the inner surface at an angle of approximately 45 degrees to the axis of handle 24 as shown. The inner most diameter of annular squeegee 26 is less than the outer diameter of endoscope shaft 4. The angle, shape, and material properties of annular squeegee 26 provide for a low friction interface between endoscope shaft 4 and annular squeegee 26, and provides a means for cleaning endoscope shaft 4 as endoscope shaft 4 is withdrawn into handle 24, endoscope cover 1. Optional annular wiping material 27 may be disposed on the interior of handle 24 as shown, and is configured to absorb residual bodily matter and lubricant not removed by annular squeegee 26 during withdrawal of endoscope shaft 4 into endoscope cover 1. Wiping material 27 may be similar to the wiping material previously described. In addition, handle 24 may comprise a radial array of linear protrusions 25 disposed on the interior of handle 24 as best shown in FIG. 9B. The inner diameter of protrusion array 25 is slightly greater than the outer diameter of endoscope shaft 4. The function of protrusion array 25, is to provide gripping surface for gripping endoscope shaft 4, with a lighter grasping force than if the inner wall of handle was configured as a solid between its outer surface and its inner surface. Handle 24, annular squeegee 26, and protrusion array 25 may be molded or cast from an elatomeric material such as silicone or urethane rubber in the configuration shown, or in a similar configuration. FIG. 9D is a cross sectional illustration of and alternative embodiment of endoscope cover handle 29. Endoscope handle 29 is similar to endoscope handle 24 described above with the addition of lubricant dispenser 30 disposed on the inner surface of handle 29 as shown. Lubricant dispenser 30 comprises lubricant bladder 32, which is filled with lubricant 31, and lubricant dispenser ports 33, disposed in an annular array at the distal end of lubricant bladder 32. Lubricant bladder 32 may be filled with lubricant 31 at the factory, or lubricant bladder 32 may be filled by the user, or a technician that prepares the endoscope cover for use. Means for filling lubricant bladder are not shown here, but may include filling lubricant bladder 32 with lubricant 31 using a hypodermic needle and syringe, or some other filling means known to those skilled in the art. Lubricant bladder 32 is configured to apply lubricant 31 to endoscope shaft 4 when the user squeezes handle 29 over handle lubricant dispensing segment 35, which is the distal segment to the right of the vertical line in the figure. Handle gripping segment 34 (to the left of the vertical line) provides a means for the user to grasp and manipulate endoscope shaft 4 using internal grippers 25 as described above.

The forgoing description and the drawings are illustrative of the invention and are not to be taken as limiting. Still other variants and rearrangements of structural parts are possible without departing from the spirit and scope of this invention and will readily present themselves to those skilled in the art.

1. An endoscope cover comprising:
an elongated, flexible sleeve sized to receive an endoscope shaft and having a length sufficient to extend beyond full length of an endoscope shaft, a proximal end portion, and a distal end portion;
a collar at the proximal end portion sized for removable engagement with the endoscope shaft; and
a handle at the distal end portion for positioning the distal end of the sleeve relative to the distal end of the endoscope shaft.

2. The endoscope cover of claim 1, wherein the collar is elastic and has an inner diameter larger than an insertion portion of the endoscope shaft but smaller than the diameter of the endoscope shaft at location of endoscope shaft engagement.

3. The endoscope cover of claim 1 wherein the handle is a tube.

4. The endoscope cover of claim 1 wherein the handle is provided with endoscopic lubricant dispenser.

5. The endoscope cover of claim 1 wherein inner surface of the handle is lined with an absorbent material.

6. The endoscope cover of claim 1 wherein an annular squeegee is disposed within the handle.

7. The endoscope cover of claim 5 wherein an annular squeegee is disposed within the handle adjacent to the absorbent material.

8. The endoscope cover of claim 1 wherein the elongated, flexible sleeve is a polyolefin tube.

9. The endoscope cover of claim 1 wherein the elongated, flexible sleeve has a length sufficient for forming a prolapsed seal over the distal end thereof.

10. The endoscope cover of claim 1 wherein the endoscope cover further comprises a removable end-cap enclosing the distal end of the sleeve.

11. A method for improving hygiene during an endoscopic procedure comprising the steps of:
a. cleaning and sanitizing an endoscope for use; then,
b. introducing an insertable portion of the endoscope shaft into the endoscope cover defined by claim 1;
c. sliding the distal end portion of the endoscope cover to expose the distal end of the endoscope shaft; and then
d. inserting the endoscope into the patient and performing an endoscopic examination or a surgical procedure.
